(19) United States
(12) Patent Application Publication
(10) Pub. No.: US 2023/0277346 A1
(43) Pub. Date: Sep. 7, 2023

(54) BIORESORBABLE TUBULAR ORGAN STENT

(71) Applicant: University of Washington, Seattle, WA (US)

(72) Inventors: James O. PARK, Seattle, WA (US); Eric J. SEIBEL, Seattle, WA (US); Abdulmalek ALOTHMAN, Seattle, WA (US); Clara BLACK, Seattle, WA (US); Brandon LOU, Seattle, WA (US); Gillian PEREIRA, Seattle, WA (US); Evan ROSS, Seattle, WA (US); Ziming YE, Seattle, WA (US)

(21) Appl. No.: 18/177,022

(22) Filed: Mar. 1, 2023

Related U.S. Application Data
(60) Provisional application No. 63/315,837, filed on Mar. 2, 2022.

(51) Int. Cl. A61F 2/915

(52) U.S. Cl. CPC A61F 2/915; A61F 2002/045; A61F 2210/0004

(57) ABSTRACT

Stent assemblies and related methods for creating an anastomosis employ a bioresorbable stent. A stent assembly includes a bioresorbable stent and one or more bioresorbable retention devices. The stent is configured for retaining a first section of a tubular organ in contact with a second section of the tubular organ to accommodate wound healing for connecting of the first and second sections of the tubular organ. A first end portion of the stent is configured to accommodate insertion into the first section of the tubular organ and inhibit removal from the first section of the tubular organ. A second end portion of the stent is configured to accommodate insertion into the second section of the tubular organ and inhibit removal from the second section of the tubular organ. The one or more retention devices are configurable to secure the first and second sections of the tubular organ to the stent.

### CROSS-REFERENCES TO RELATED APPLICATIONS

This application claims the benefit of U.S. Provisional Application No. 63/315,837 filed Mar. 2, 2022, the entire contents of which are hereby incorporated for all purposes in their entirety.

### BACKGROUND

The financial burden of gastrointestinal (GI) diseases in the United States is among one of the highest at $136 billion/year, which is more than the $113 billion/year spent on heart diseases. There are approximately 700,000 gastrointestinal anastomotic surgeries performed worldwide. An anastomosis is a surgical connections between two tubular organs. An anastomosis may be created following the removal of a section of a tubular organ that includes pathologic entities (e.g., tumors). Current methods for creating a GI anastomosis include surgically connecting the remaining sections of the intestine via suturing and/or stapling via laparoscopy or laparotomy. Creating a GI anastomosis using such current methods, however, can be expensive, time consuming, invasive, and result in a high rate of complications.

A major complication that may occur following gastrointestinal anastomotic procedure is leaking of GI content from the anastomosis. Common complications resulting from these leaks include surgical site infection, intraabdominal abscesses, ulcers, gastrointestinal stricture, pneumonia, and septic shock. Leaking of GI content from an anastomosis occurs in about 6% to 30% of cases. Whether an anastomosis leaks is believed to depend, in part, on patient risk factors as well as anastomosis type. In up to 60% of cases, a leaking GI anastomosis requires surgical repair. Complications arising from a leaking GI anastomosis are often especially detrimental due to the bacterial content in the fluids flowing through the GI tract.

### BRIEF SUMMARY

The following presents a simplified summary of some embodiments of the invention to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Bioresorbable tubular organ stent assemblies for creating an anastomosis that connects sections of a tubular organ (e.g., following removal of a section of the tubular organ), and related methods, employ a stent configured to maintain contact between the sections of the tubular organ to promote fusing of the sections via healing. In many embodiments, the tubular stent assembly is configured to isolate the interfaced portions of the sections of the tubular organ from the contents of the tubular organ to inhibit leakage from the anastomosis and better promote fusing of the sections via healing. While the bioresorbable tubular organ stent assemblies and related methods for creating an anastomosis can be adapted for use in creating many different types of an anastomosis (e.g., end-to-end, side-to-side, end-to-side, arterioarterial, venovenous, arteriovenous, circulatory, ductal, stomach, intestinal, etc.), they may be especially beneficial for creating a GI anastomosis due to reduced rates of leakage of GI tract contents and associated reduction in related complications. The bioresorbable tubular organ stent assemblies and related methods for creating an anastomosis may be adapted for use in connecting any suitable tubular structure in a patient (e.g., human, non-human) such as the GI tract (intestines, biliary ducts, etc.), uterine tract (e.g., fallopian tube), urinary tract (ureter), and cardiovascular arteries and veins.

Thus, in many embodiments, a bioresorbable tubular organ stent assembly includes a bioresorbable stent and one or more bioresorbable retention devices. The stent is configured for retaining a first section of a tubular organ in contact with a second section of the tubular organ to accommodate joining of the first section and the second section. A first end portion of the stent is configured to accommodate insertion into the first section of the tubular organ and inhibit removal from the first section of the tubular organ. A second end portion of the stent is configured to accommodate insertion into the second section of the tubular organ and inhibit removal from the second section of the tubular organ. The stent is bioresorbable. The one or more retention devices are configurable to secure the first section and the second section to the stent. Each of the one or more retention devices is bioresorbable.

The bioresorbable tubular organ stent assembly can be used to connect sections of any suitable tubular organ. For example, in many embodiments, the tubular organ is an intestine. In many embodiments, the stent includes a tubular outer wall that defines a lumen configured for fluidly coupling the first section of the tubular organ and the second section of the tubular organ. The tubular outer wall can be configured to block flow of contents of the tubular organ to interfaced portions of the first section and the second section. In many embodiments, the first end portion of the stent includes one or more first end portion retention features and the second end portion of the stent includes one or more second end portion retention features. The one or more first end portion retention features can be configured to interface with the first section of the tubular organ and shaped to accommodate insertion of the first end portion of the stent into the first section of the tubular organ and inhibit removal of the first end portion of the stent from the first section of the tubular organ. The one or more second end portion retention features can be configured to interface with the second section of the tubular organ and shaped to accommodate insertion of the second end portion of the stent into the second section of the tubular organ and inhibit removal of the second end portion of the stent from the second section of the tubular organ. In some embodiments, the one or more first end portion retention features include first end portion protruding barbs and the one or more second end portion retention features include second end portion protruding barbs.

The bioresorbable tubular organ stent assembly can be configured to be implantable using a minimally invasive surgical procedure. For example, the stent can be an expandable stent configured to be expandable from a collapsed configuration to an expanded configuration. The collapsed configuration can be configured to accommodate insertion of the stent through a surgical port. The expanded configuration can be configured for retaining the first section in contact with the second section. In some embodiments, the expandable stent includes an expandable outer membrane configured to block flow of contents of the tubular organ to interfaced portions of the first section of the tubular organ and the second section of the tubular organ. In some embodiments, the expandable stent includes stent links. Each of the stent links can be pivotally coupled with each of an adjacent two of the stent links via pivot joints configured to accommodate expansion of the expandable stent from the collapsed configuration to the expanded configuration and block reconfiguration of the expandable stent from the expanded configuration to the collapsed configuration. Each of one or more of the pivot joints can include a ratchet mechanism configured to accommodate pivoting between a pair of the stent links joined by the pivot j oint in a first rotational direction and block pivoting between the pair of the stent links in a second rotational direction opposite to the first rotational direction. Each of the stent links can form a respective portion of the first end portion of the stent and a respective portion of the second end portion of the stent. Each of stent links can include one or more first end portion retention features and one or more second end portion retention features. The one or more first end portion retention features can be configured to interface with the first section of the tubular organ and shaped to accommodate insertion of the first end portion of the expandable stent into the first section of the tubular organ and inhibit removal of the first end portion of the expandable stent from the first section of the tubular organ. The one or more second end portion retention features can be configured to interface with the second section of the tubular organ and shaped to accommodate insertion of the second end portion of the expandable stent into the second section of the tubular organ and inhibit removal of the second end portion of the expandable stent from the second section of the tubular organ. In some embodiments, the one or more first end portion retention features include first end portion protruding barbs and the one or more second end portion retention features include second end portion protruding barbs.

In many embodiments, the one or more retention devices are operable to clamp the first section and the second section to an exterior surface of the stent. In some embodiments, at least one of the one or more retention devices includes a clamping strap and a ratchet mechanism for adjusting a circumference of the clamping strap. In some embodiments, the one or more retention devices includes at least one of a first clamp and a second clamp. The first clamp can include a first clamping strap and a first ratchet mechanism for adjusting a circumference of the first clamping strap. The second clamp can include a second clamping strap and a second ratchet mechanism for adjusting a circumference of the second clamping strap. In some embodiments, the one or more retention devices include a helical clamping coil configured to clamp the first section of the tubular organ and the second section of the tubular organ to the exterior surface of the stent.

The stent can include any suitable therapeutic compound. For example, in some embodiments, the stent can include an antibacterial coating. In some embodiments, the antibacterial coating includes one or more of cellulose, silver, or a resorbable polymer coating infused with an antibiotic compound.

The stent can include a porous structure. In some embodiments, the porous structure includes interconnected pores.

The stent and the one or more retention devices can be made from one or more suitable bioresorbable materials. For example, the stent and the one or more retentions devices can include one or more of magnesium (Mg), zinc (Zn), iron (Fe), tungsten (W), molybdenum (Mo), polylactic acid (PLA), polyglycolide (PGA), poly(lactic-co-glycolic acid) (PLGA), polyethylene glycol (PEG), polycaprolactone (PCL), poly(glycerol sebacate) (PGS), alginate (all forms), cellulose (all forms), chitosan (all forms), hyaluronic acid (human and animal derived), or collagen (human and animal derived types 1, 2, 3, 4, and 5.

In many embodiments, a method of connecting sections of a tubular organ employs a bioresorbable stent. For example, a method of connecting sections of a tubular organ includes inserting a first end portion of a bioresorbable stent into an open end of a first section of the tubular organ, inserting a second end portion of the bioresorbable stent into an open end of a second section of the tubular organ, and securing the first section and the second section to the bioresorbable stent.

In many embodiments, an intestinal stent assembly include an intestinal stent and one or more retention devices. The intestinal stent is configured for retaining a first section of an intestine in contact with a second section of the intestine to accommodate joining of the first section and the second section via healing. A first end portion of the intestinal stent is configured to accommodate insertion into the first section of the intestine and inhibit removal from the first section of the intestine. A second end portion of the intestinal stent is configured to accommodate insertion into the second section of the intestine and inhibit removal from the second section of the intestine. The intestinal stent is made from one or more bioresorbable materials. The one or more retention devices are configurable to secure the first section of the intestine and the second section of the intestine to the intestinal stent. Each of the one or more retention devices is made from one or more bioresorbable materials.

For a fuller understanding of the nature and advantages of the present invention, reference should be made to the ensuing detailed description and accompanying drawings.

### BRIEF DESCRIPTION OF THE DRAWINGS

FIG. 1 illustrates a tubular organ anastomosis formed via a bioresorbable stent assembly, in accordance with embodiments.

FIG. 2a, FIG. 2b, and FIG. 2c illustrate a bioresorbable stent that can be employed in the bioresorbable stent assembly of FIG. 1.

FIG. 3a, FIG. 3b, and FIG. 3c illustrate a bioresorbable clamp that can be employed in the bioresorbable stent assembly of FIG. 1.

FIG. 4 is a simplified schematic flow-chart of a method of connecting sections of an intestine, in accordance with embodiments.

FIG. 5 illustrates a tubular organ anastomosis formed via another bioresorbable stent assembly, in accordance with embodiments.

FIG. 6 illustrates a tubular organ anastomosis formed via another bioresorbable stent assembly, in accordance with embodiments.

FIG. 7 is a simplified schematic flow-chart of a method of connecting sections of a tubular organ, in accordance with embodiments.

FIG. 8a, FIG. 8b, FIG. 8c, and FIG. 8d illustrate expanded configurations of embodiments of an expandable bioresorbable stent that can be employed in the bioresorbable stent assembly of FIG. 1.

FIG. 9a, FIG. 9b, and FIG. 9c, illustrate collapsed configurations of the expandable bioresorbable stent embodiments of FIG. 8a, FIG. 8b, FIG. 8c, and FIG. 8d.

FIG. 10a and FIG. 10b illustrate a one-directional ratcheting connection between adjacent links of the expandable bioresorbable stent embodiments of FIG. 8a, FIG. 8b, FIG. 8c, and FIG. 8d.

FIG. 11a and FIG. 11b illustrate tubular organ retention features of a representative link of the expandable bioresorbable stent embodiments of FIG. 8a, FIG. 8b, FIG. 8c, and FIG. 8d.

FIG. 12 illustrates a representative pair of the links of the expandable bioresorbable stent embodiments of FIG. 8a, FIG. 8b, FIG. 8c, and FIG. 8d.

FIG. 13 is a simplified schematic flow-chart of a method of connecting sections of a tubular organ using an expandable bioresorbable stent, in accordance with embodiments.

FIG. 14 shows micrographs of a porous structure with interconnected pores that can be employed in a bioresorbable stent, in accordance with embodiments.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

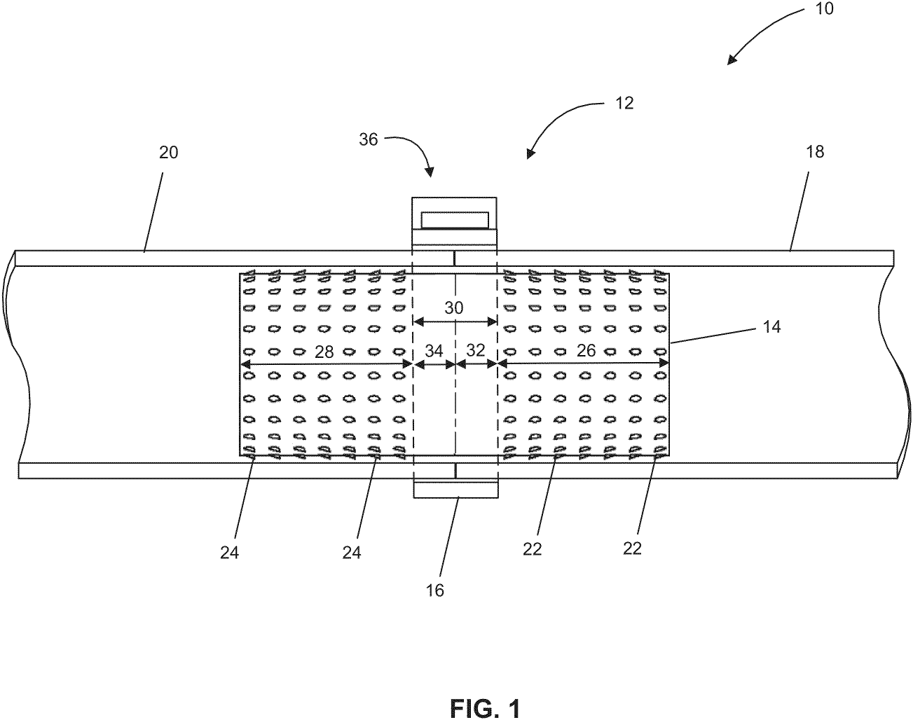

Referring now to the drawings wherein like reference numerals are used to identify identical components in the various views, FIG. 1 illustrates a tubular organ anastomosis 10 formed via a bioresorbable stent assembly 12, in accordance with embodiments. The bioresorbable stent assembly 12 includes a bioresorbable stent 14 and one or more bioresorbable retention devices 16. The bioresorbable stent 14 is configured for retaining a first section 18 of a tubular organ in contact with a second section 20 of the tubular organ following removal of a resected section of the tubular organ to accommodate wound healing for rejoining of the first section 18 and the second section 20. The stent 14 includes first end unidirectional retention features 22 and second end unidirectional retention features 24. The first end retention features 22 are distributed on a first end portion 26 of the stent 14 and are configured to accommodate insertion of the first end portion 26 into the open end of the first section 18 of the tubular organ and inhibit movement of the first end portion 26 of the stent 14 out of the first section 18. The second end retention features 22 are distributed on a second end portion 28 of the stent 14 and are configured to accommodate insertion of the second end portion 28 into the open end of the second section 20 of the tubular organ and inhibit movement of the second end portion 28 of the stent 14 out of the second end section 20. In the illustrated embodiment, the retention features 22, 24 are configured as an array of protruding barbs that are sloped and oriented to accommodate insertion of the stent 14 into the first and second sections 18, 20 of the tubular organ and resist removal of the stent 14 from the first and second sections 18, 20 of the tubular organ. The stent 14 includes a central portion 30. A first portion 32 of the central portion 30 is configured to be inserted into the open end of the first section 18 of the tubular organ. A second portion 34 of the central portion 30 is configured to be inserted into the open end of the second section 20 of the tubular organ so as to interface end surfaces of the first and second sections 18, 20 over the central portion 30. In the illustrated embodiment, the one or more retention devices 16 are configured as an adjustable band clamp 16 with a ratchet mechanism 36 for selectively reducing the circumference of the band clamp 16 around the interfaced portions of the first and second sections 18, 20 of the tubular organ to secure the first and second sections 18, 20 to the stent 14 to maintain contact between the end surfaces of the first and second sections 18, 20 in support of healing induced fusion of the first and second sections 18, 20, as well to help block flow of contents of the tubular organ to the interfaced portions of the first and second sections 18, 20. In many embodiments, the band clamp 16 is formed from a suitable bioresorbable material. In some embodiments, the band clamp 16 is made from an elastic bioresorbable material. In some embodiments, the band clamp 16 is made from an elastic bioresorbable material and lacks a mechanism (e.g., a ratcheting mechanism) for adjusting the circumference of the band claim 16.

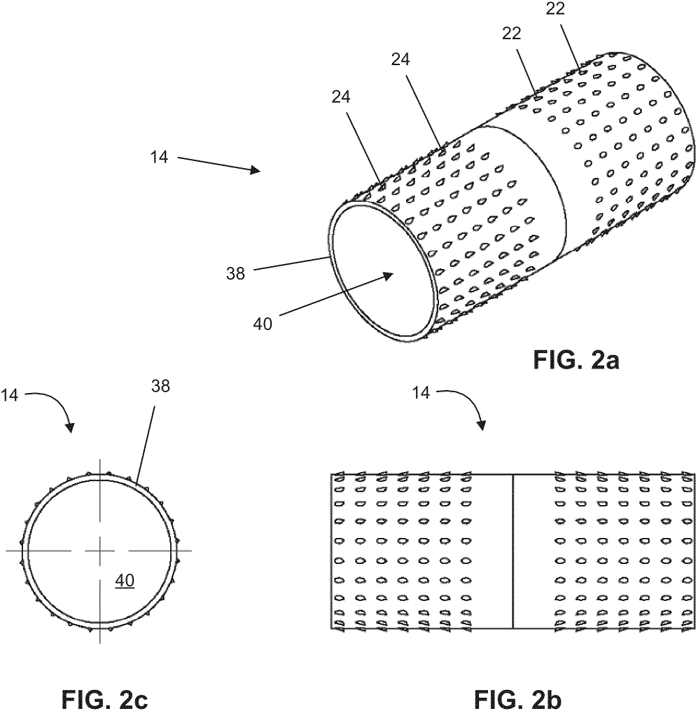

FIG. 2a shows an isometric view of the bioresorbable stent 14. FIG. 2b shows a side view of the stent 14. FIG. 2c shows an end view of the stent 14. In the illustrated embodiment, the stent 14 includes a cylindrical wall 38 that defines a lumen 40 configured for fluidly coupling the first and second sections 18, 20 of the tubular organ. The cylindrical wall 38 is configured to interface with the first and second sections 18, 20 so as to block flow of contents of the tubular organ around the exterior of the stent 14 to protect the interfaced end portions of the first and second sections 18, 20 of the tubular organ from exposure to the contents of the tubular organ. The cylindrical wall 38 is also configured to provide a continuous barrier to block passage of the contents of the tubular organ directly through the cylindrical wall to protect the interfaced end portions of the first and second sections 18, 20 of the tubular organ from exposure to the contents of the tubular organ. The stent 14 can be made from one or more suitable bioresorbable materials, such as one or more of magnesium (Mg), zinc (Zn), iron (Fe), tungsten (W), molybdenum (Mo), polylactic acid (PLA), polyglycolide (PGA), poly(lactic-co-glycolic acid) (PLGA), polyethylene glycol (PEG), polycaprolactone (PCL), poly(glycerol-sebacate) (PGS), alginate (all forms), cellulose (all forms), chitosan (all forms), hyaluronic acid (human and animal derived), or collagen (human and animal derived types 1, 2, 3, 4, and 5). In many embodiments, the stent 14 is configured to resorb at a suitable rate to selected to provide a suitable time span (e.g., 1 week to 5 or more years) during which the first and section sections 18, 20 are supported by the stent 14 to produce sufficient fusing of the first and second sections 18, 20 prior to the stent 14 being resorbed to an extent that the stent 14 no longer provides sufficient support to the first and sections 18, 20 of the tubular organ.

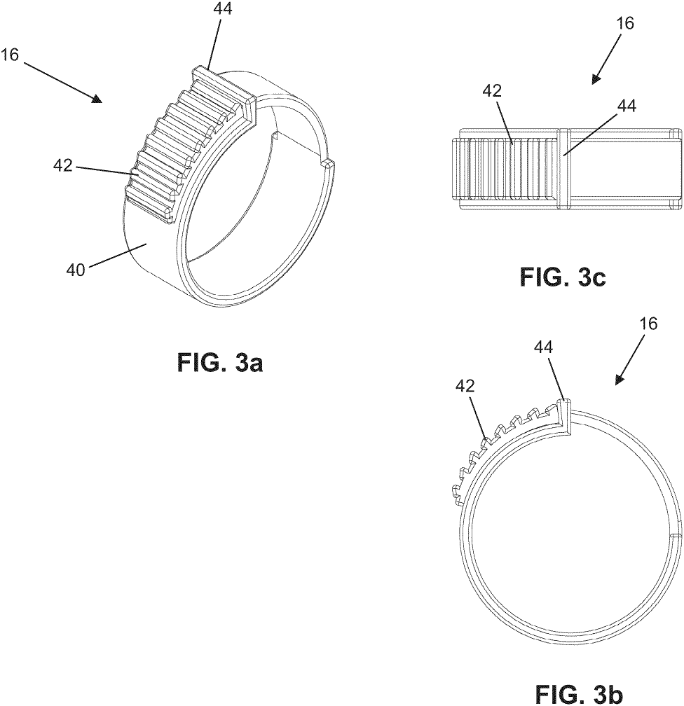

FIG. 3a shows an isometric view of the band clamp 16. FIG. 3b, shows a side view of the band clamp 16. FIG. 3c shows an end view of the band clamp 16. The band clamp 16 includes a strap 40 with protruding ratchet features 42 and an anchor feature 44. The circumference of the band clamp 16 can be selectively reduced via pulling of the end of the strap 40 through the anchor feature 44 to so as to select which of the ratchet features 42 is engaged with the anchor feature 44, thereby enabling the ability to selectively adjust the amount of clamping pressure applied to the first and second sections 18, 20 by the band clamp 16. The band clamp 16 can be made from one or more suitable bioresorbable materials, such as one or more of magnesium (Mg), zinc (Zn), iron (Fe), tungsten (W), molybdenum (Mo), polylactic acid (PLA), polyglycolide (PGA), poly(lactic-co-glycolic acid) (PLGA), polyethylene glycol (PEG), polycaprolactone (PCL), poly(glycerol-sebacte) (PGS), alginate (all forms), cellulose (all forms), chitosan (all forms), hyaluronic acid (human and animal derived), or collagen (human and animal derived types 1, 2, 3, 4, and 5). In many embodiments, the band clamp 16 is configured to resorb at a suitable rate selected to provide a suitable time span (e.g., 1 week to 5 or more years) during which the first and section sections 18, 20 are clamped to the stent 14 by the band clamp 16 to support sufficient fusing of the first and second sections 18, 20 prior to the band clamp 16 being resorbed.

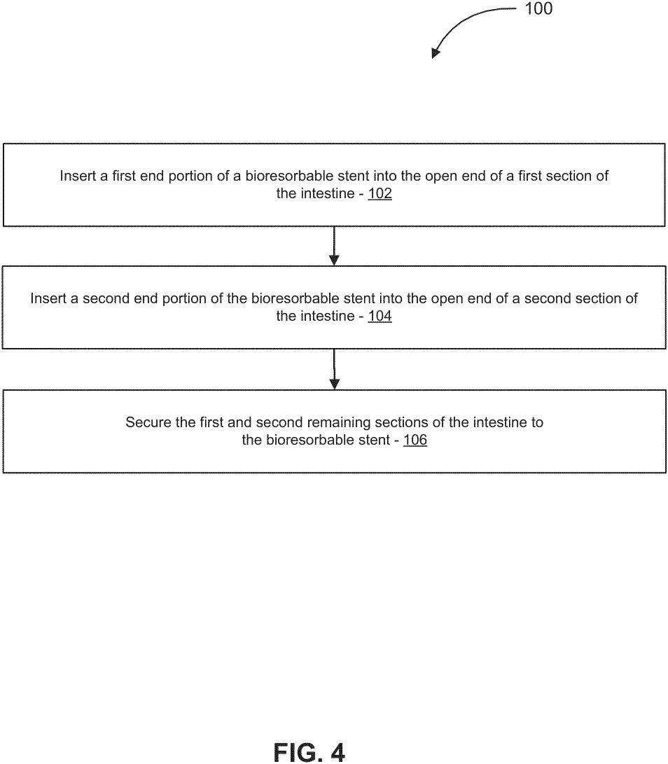

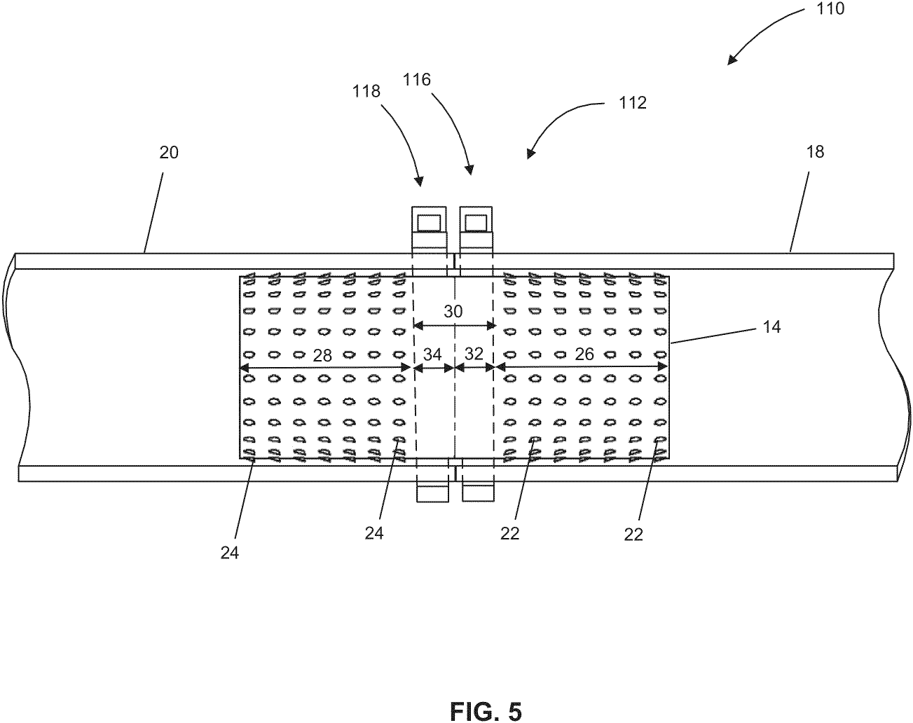

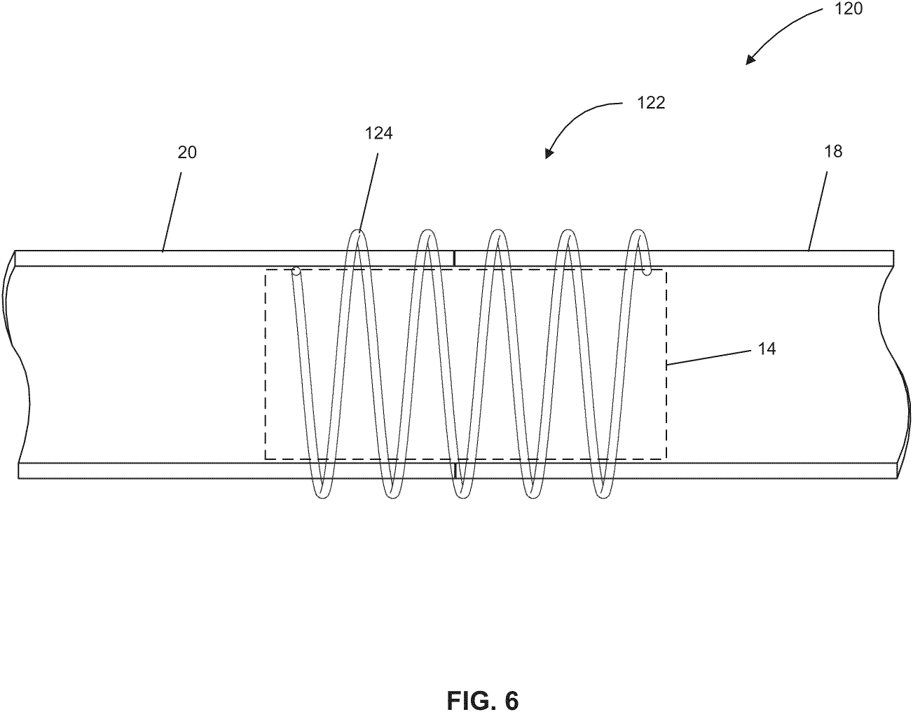

FIG. 4 is a simplified schematic flow-chart of a method 100 of connecting sections of an intestine, in accordance with embodiments. The method 100 can be practiced using any suitable bioresorbable stent assembly, such as the bioresorbable stent assemblies described herein. In act 102, a first end portion of a bioresorbable stent is inserted into the open end of a first section of the intestine. In act 104, a second end portion of the bioresorbable stent is inserted into the open end of a second section of the intestine to bring an end surface of the second section into contact with an end surface of the first section over a central portion of the bioresorbable stent. In act 106, the first and second sections of the intestine are secured to the bioresorbable stent to maintain contact between the ends of the first and second sections of the intestine. Any suitable approach can be used to secure the first and second sections of the intestine to the bioresorbable stent, such as via clamping via the band clamp 16, band claims 116, 118 (shown in FIG. 5), and/or a clamping coil 124 (shown in FIG. 6) as described herein.

FIG. 5 illustrates a tubular organ anastomosis 110 formed via another bioresorbable tubular organ stent assembly 112, in accordance with embodiments. The bioresorbable stent assembly 112 is configured the same as the stent assembly 12 except for employing two band clamps 116, 118. Each of the band clamps 116, 118 can be configured the same as the band clamp 16, but with a reduced width. The use of the two band clamps 116, 118 instead of the single band clamp 16 can be used to facilitate insertion of the second end portion 28 of the stent 14 into the second section 20 of the tubular organ via first clamping of the first section 18 of the tubular organ to the stent 14 via the band clamp 116 and then grasping the band claim 116 during installation of the second end portion 28 of the stent 14 into the second section 20 of the tubular organ. One or both of the band clamps 116, 118 can include one or more grasping features to facilitate insertion of the stent 14 into the tubular organ section 20.

FIG. 6 illustrates a tubular organ anastomosis 120 formed via another bioresorbable stent assembly 122, in accordance with embodiments. The bioresorbable stent assembly 122 is configured the same as the stent assembly 12 except for employing a helical clamping coil 124 instead of the band clamp 16. The helical clamping coil 124 is configured to provide clamping of the first and second sections 18, 20 of the tubular organ to the stent 14 along the length of the stent 14 to help better block flow of contents of the tubular organ along the exterior surface of the stent 14 from reaching the interfaced end portions of the first and second sections 18, 20 of the tubular organ. The helical clamping coil 124 can be fabricated by coiling an elongated, narrow strip of bioresorbable metal or plastic into a helical shape. The helical clamping coil 124 is configured for placement on the outside of the first and second sections 18, 20 of the tubular organ to secure the first and second sections 18, 20 to the stent 14. The diameter and pitch of each turn of the clamping coil 124 can be uniform. The clamping coil 124 can be configured to be elastic so that the clamping coil 124 will return to its original shape after being stretched or compressed, thereby accommodating insertion of the clamping coil 124 through a surgical access port (e.g., a laparoscopic port). The clamping coil 124 can be configured to extend over almost the entire length of the stent 14 to seal the first and sections 18, 20 to the stent 14 along almost the entire length of the stent 14. In some embodiments, two of the clamping coils 124 are employed in conjunction with the band clamp 116 to individually seal each of the first and second sections 18, 20 of the tubular organ to the respective interfacing portions of the stent 14.

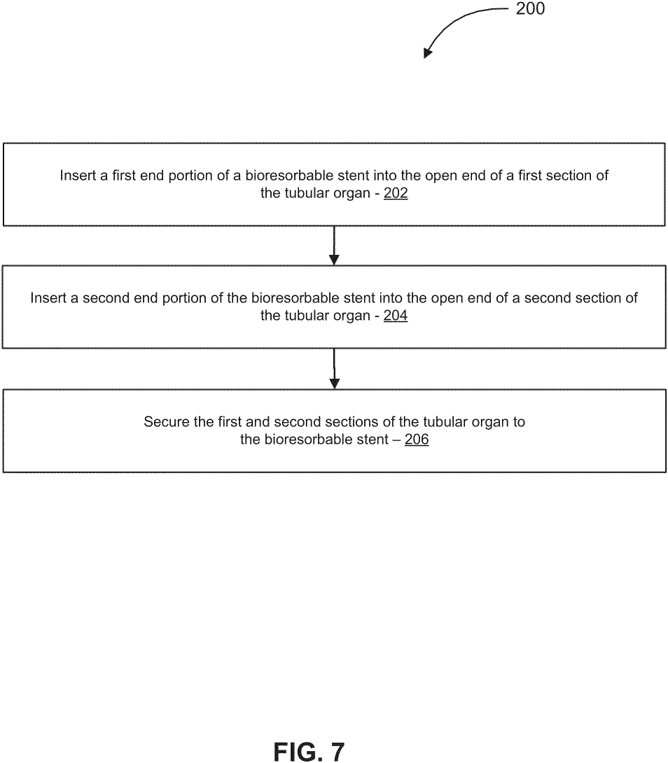

FIG. 7 is a simplified schematic flow-chart of a method 200 of connecting sections of a tubular organ, in accordance with embodiments. The method 200 can be practiced using any suitable bioresorbable stent assembly, such as the bioresorbable stent assemblies described herein. In act 202, a first end portion of a bioresorbable stent is inserted into the open end of a first section of the tubular organ. In act 204, a second end portion of the bioresorbable stent is inserted into the open end of a second section of the tubular organ to bring an end surface of the second section into contact with an end surface of the first section over a central portion of the bioresorbable stent. In act 206, the first and second sections of the tubular organ are secured to the bioresorbable stent to maintain contact between the end portions of the first and second sections of the tubular organ. Any suitable approach can be used to secure the first and second sections of the tubular organ to the bioresorbable stent, such as via clamping via the band clamp 16, the band claims 116, 118, and/or the clamping coil(s) 124 as described herein.

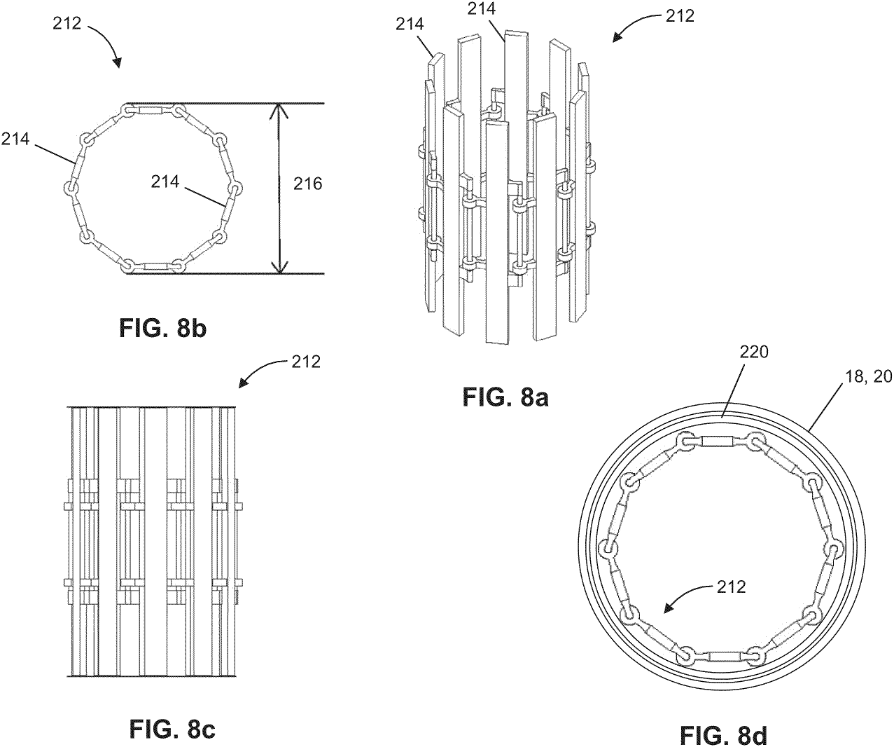

FIG. 8a, FIG. 8b, FIG. 8c, and FIG. 8d illustrate an expanded configuration of embodiments of an expandable bioresorbable stent 212 that can be employed in the bioresorbable stent assemblies 12, 112, 122 described herein. The stent 212 is expandable from a collapsed configuration to the expanded configuration. The collapsed configuration of the stent 212 accommodates insertion of the stent 212 through a surgical access port. The expanded configuration of the stent 212 is configured for retaining the first section 18 of the tubular organ in contact with the second section 20 of the tubular organ. In the illustrated embodiment, the expandable stent 212 includes ten pivotally coupled links 214. The stent 212 can, however, include any suitable number of the links 214. In the expanded configuration, the expandable stent 212 has an expanded diameter 216. In the embodiment shown in FIG. 8d, the stent 212 includes an expandable outer membrane or wall 220 configured to block flow of contents of the tubular organ to interfaced portions of the first and second sections 18, 20 of the tubular organ.

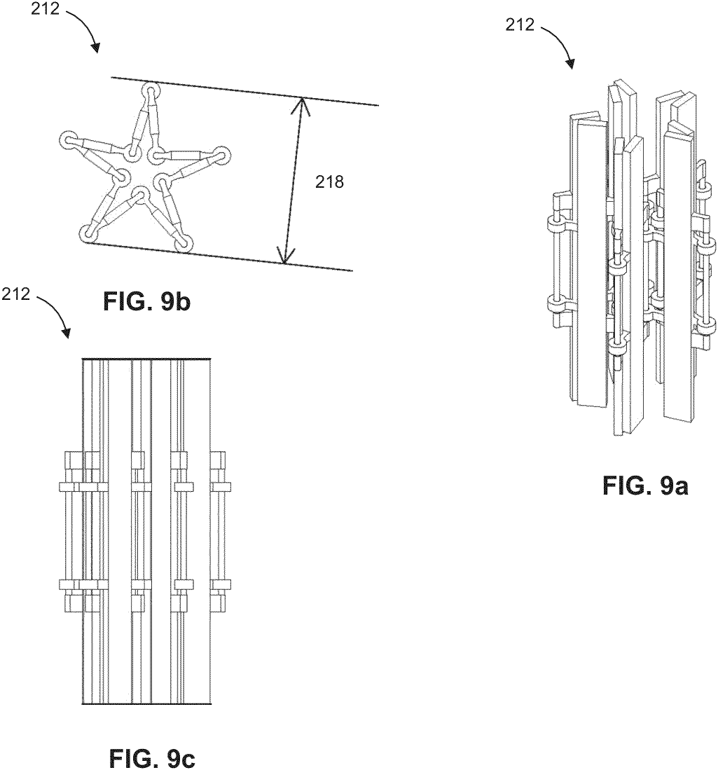

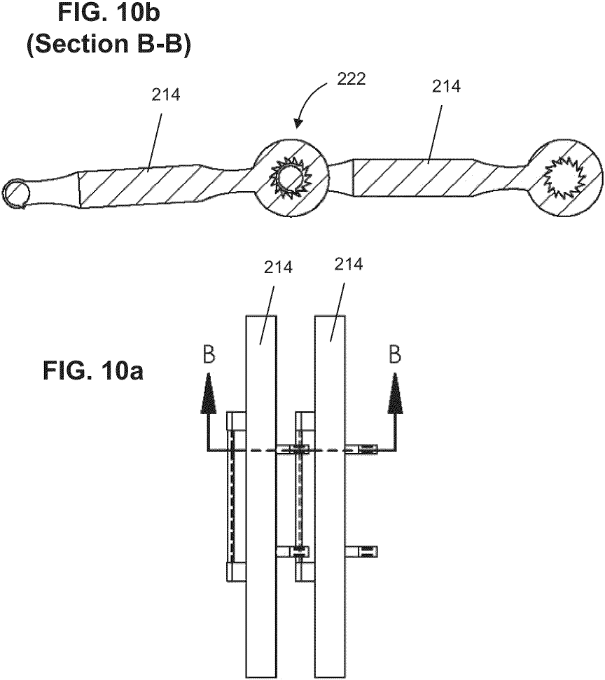

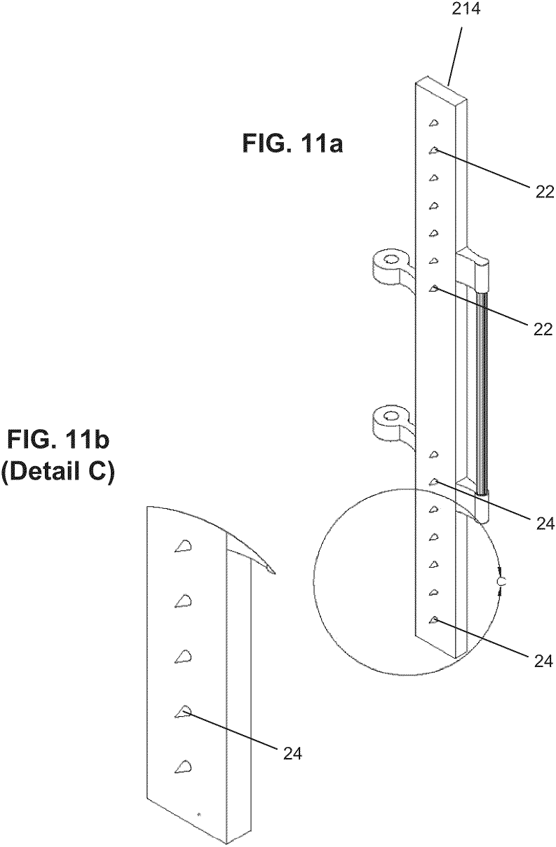

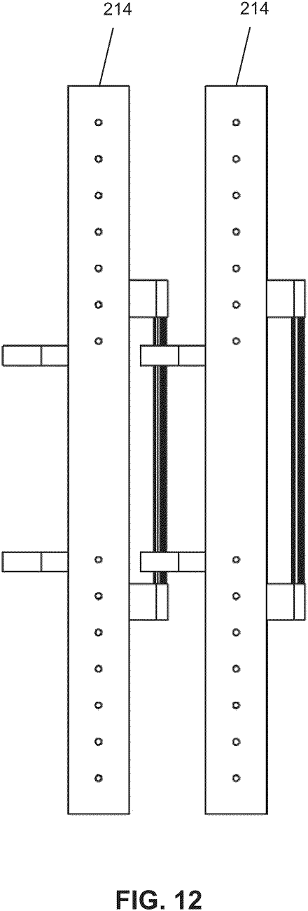

FIG. 9a, FIG. 9b, and FIG. 9c, illustrate the collapsed configuration of the expandable stent 212. In the collapsed configuration, the expandable stent has a collapsed diameter 218, which in the illustrated embodiments is approximately 55 percent of the expanded diameter 216. The smaller collapsed diameter 218 reduces the size of a surgical access port that can be used to introduce the expandable stent 212 into a patient during a minimally invasive surgical procedure for forming an anastomosis. FIG. 10a and FIG. 10b illustrate a one-directional ratcheting connection between adjacent links 214 of the expandable stent 212. FIG. 11a and FIG. 11b illustrate tubular organ retention features of a representative link 214 of the expandable bioresorbable stent 212. FIG. 12 illustrates a representative pair of the links 214.

Each of the stent links 214 is pivotally coupled with each of an adjacent two of the stent links 214 via pivot joints 222 configured to accommodate expansion of the stent 212 from the collapsed configuration to the expanded configuration and block reconfiguration of the stent 212 from the expanded configuration to the collapsed configuration. In the illustrated embodiment, each of the pivot joints 222 includes a ratchet mechanism configured to accommodate pivoting between a pair of the stent links 214 joined by the pivot joint 222 in a first rotational direction and block pivoting between the pair of the stent links 214 in a second rotational direction opposite to the first rotational direction. Each of the stent links 214 forms a respective portion of the first end portion of the stent and a respective portion of the second end portion of the stent. Each of the stent links 214 includes one or more of the first end portion retention features 26 and one or more of the second end portion retention features 28.

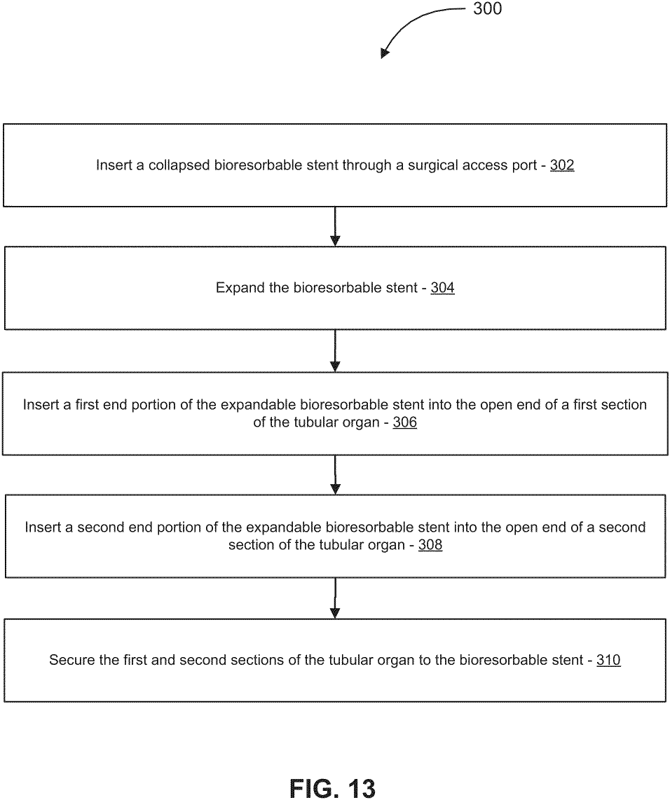

FIG. 13 is a simplified schematic flow-chart of a method 300 of connecting sections of a tubular organ using an expandable bioresorbable stent, in accordance with embodiments. The method 300 can be practiced using any suitable bioresorbable stent assembly, such as the bioresorbable stent assemblies described herein. In act 302, an expandable stent in a collapsed configuration is inserted through a surgical access port (e.g., a laparoscopic port). In act 304, following the insertion of the expandable stent in act 302, the expandable stent is expanded from the collapsed configuration to an expanded configuration. In act 306, a first end portion of the expandable bioresorbable stent is inserted into the open end of a first section of the tubular organ. In act 308, a second end portion of the bioresorbable stent is inserted into the open end of a second section of the tubular organ to bring an end surface of the second section into contact with an end surface of the first section over a central portion of the expandable bioresorbable stent. The expansion of the expandable stent (act 304) can be accomplished before act 306 or after act 306 and before act 308. Any suitable approach (e.g., manual manipulation, expansion via an expandable balloon) can be used to expand the stent 212. In act 310, the first and second sections of the tubular organ are secured to the expanded bioresorbable stent to maintain contact between the interfaced end portions of the first and second sections of the tubular organ. Any suitable approach can be used to secure the first and second sections of the tubular organ to the bioresorbable stent, such as via clamping via the band clamp 16, the band claims 116, 118, and or the clamping coils 124 as described herein.

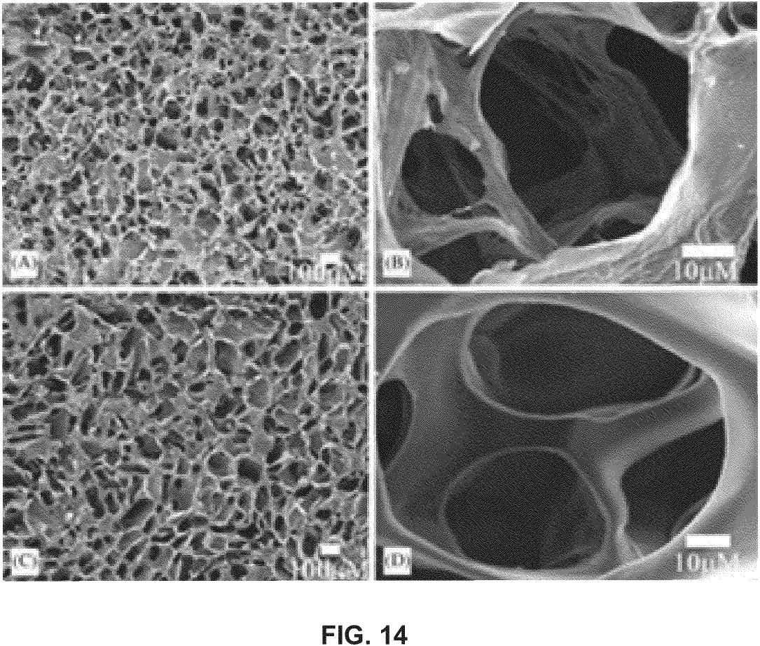

FIG. 14 shows micrographs of a porous structure with interconnected pores that can be employed in or on the surface of the bioresorbable stents 12, 112, 122, 212. Any suitable material, such as one or more of magnesium (Mg), zinc (Zn), iron (Fe), tungsten (W), molybdenum (Mo), PLA, PGA, PLGA, PEG, PCL, PGS, alginate (all forms), cellulose (all forms), chitosan (all forms), hyaluronic acid (human and animal derived), or collagen (human and animal derived types 1, 2, 3, 4, and 5, can be used to form the bioresorbable stents 12, 112, 122, 212 to have a porous structure with interconnected pores. Embodiments of the bioresorbable stents 12, 112, 122, 212 with high porosity and highly interconnected pore structures may help facilitate gas and nutrient exchange to enhance cell proliferation. The pores may also be beneficial for tissue vascularization and the formation of new tissues. The pores sizes and connectivity can be selected to restrict passage of infectious agents such as bacteria for a period of time before breaking down in a controlled bio-resorption process.

The bioresorbable stents 12, 112, 122, 212 can include one or more therapeutic compounds and configured to elute the one or more therapeutic compounds through direct tissue contact and/or during resorption of the stent. Some non-limiting examples of therapeutic compounds that could be included within the stents 12, 112, 122, 212 are anti-inflammatory compounds, anti-coagulant compounds, antimicrobial compounds, chemotherapy compounds, and/or pain relief compounds.

The bioresorbable stents 12, 112, 122, 212 can be configured to have any suitable length, outer diameter, pre-expanded outer diameter, expanded outer diameter, barb height, and barb area length. Some non-limiting examples of suitable dimensional ranges include stent length in a range from 30 mm to 100 mm, outer diameter in a range of 10 mm to 60 mm, pre-expanded outer diameter in a range of 5 mm to 30 mm, expanded outer diameter in a range from 20 mm to 60 mm, barb height (base to tip) in a range from 0 mm to 1 mm, and bar area length in a range from 100 mm to 220 mm.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A bioresorbable tubular organ stent assembly comprising:
a stent configured for retaining a first section of a tubular organ in contact with a second section of the tubular organ to accommodate joining of the first section and the second section, wherein a first end portion of the stent is configured to accommodate insertion into the first section and inhibit removal from the first section, wherein a second end portion of the stent is configured to accommodate insertion into the second section and inhibit removal from the second section, and wherein the stent is bioresorbable; and
one or more retention devices configurable to secure the first section and the second section to the stent, wherein each of the one or more retention devices is bioresorbable.

2. The bioresorbable tubular organ stent assembly of claim 1, wherein the tubular organ is an intestine.

3. The bioresorbable tubular organ stent assembly of claim 1, wherein the stent comprises a tubular outer wall that defines a lumen configured for fluidly coupling the first section and the second section.

4. The bioresorbable tubular organ stent assembly of claim 3, wherein the tubular outer wall is configured to block flow of contents of the tubular organ to interfaced portions of the first section and the second section.

5. The bioresorbable tubular organ stent assembly of claim 3, wherein:
the first end portion comprises one or more first end portion retention features;
the second end portion comprises one or more second end portion retention features;
the one or more first end portion retention features are configured to interface with the first section and shaped to accommodate insertion of the first end portion into the first section and inhibit removal of the first end portion from the first section; and
the one or more second end portion retention features are configured to interface with the second section and shaped to accommodate insertion of the second end portion into the second section and inhibit removal of the second end portion from the second section.

6. The bioresorbable tubular organ stent assembly of claim 5, wherein:
the one or more first end portion retention features comprise first end portion protruding barbs; and
the one or more second end portion retention features comprise second end portion protruding barbs.

7. The bioresorbable tubular organ stent assembly of claim 1, wherein:
the tubular organ is an intestine;
the stent is expandable from a collapsed configuration to an expanded configuration;
the collapsed configuration accommodates insertion of the stent through a surgical port; and
the expanded configuration is configured for retaining the first section in contact with the second section.

8. The bioresorbable tubular organ stent assembly of claim 7, wherein the stent comprises an expandable outer membrane configured to block flow of contents of the tubular organ to interfaced portions of the first section and the second section.

9. The bioresorbable tubular organ stent assembly of claim 7, wherein:
the stent comprises stent links; and
each of the stent links is pivotally coupled with each of an adjacent two of the stent links via pivot joints configured to accommodate expansion of the stent from the collapsed configuration to the expanded configuration and block reconfiguration of the stent from the expanded configuration to the collapsed configuration.

10. The bioresorbable tubular organ stent assembly of claim 9, wherein each of one or more of the pivot joints comprises a ratchet mechanism configured to accommodate pivoting between a pair of the stent links joined by the pivot joint in a first rotational direction and block pivoting between the pair of the stent links in a second rotational direction opposite to the first rotational direction.

11. The bioresorbable tubular organ stent assembly of claim 9, wherein:
each of the stent links forms a respective portion of the first end portion of the stent; and
each of the stent links forms a respective portion of the second end portion of the stent.

12. The bioresorbable tubular organ stent assembly of claim 11, wherein:
each of the stent links comprises one or more first end portion retention features;
each of the stent links comprises one or more second end portion retention features;
the one or more first end portion retention features are configured to interface with the first section and shaped to accommodate insertion of the first end portion into the first section and inhibit removal of the first end portion from the first section; and
the one or more second end portion retention features are configured to interface with the second section and shaped to accommodate insertion of the second end portion into the second section and inhibit removal of the second end portion from the second section.

13. The bioresorbable tubular organ stent assembly of claim 12, wherein:
the one or more first end portion retention features comprise first end portion protruding barbs; and
the one or more second end portion retention features comprise second end portion protruding barbs.

14. The bioresorbable tubular organ stent assembly of claim 1, wherein the one or more retention devices are operable to clamp the first section and the second section to an exterior surface of the stent.

15. The bioresorbable tubular organ stent assembly of claim 14, wherein at least one of the one or more retention devices comprise a clamping strap and a ratchet mechanism for adjusting a circumference of the clamping strap.

16. The bioresorbable tubular organ stent assembly of claim 14, wherein the one or more retention devices comprise at least one of:
a first clamp comprising a first clamping strap and a first ratchet mechanism for adjusting a circumference of the first clamping strap; and
a second clamp comprising a second clamping strap and a second ratchet mechanism for adjusting a circumference of the second clamping strap.

17. The bioresorbable tubular organ stent assembly of claim 14, wherein the one or more retention devices comprise a helical clamping coil configured to clamp the first section and the second section to the exterior surface of the stent.

18. The bioresorbable tubular organ stent assembly of claim 1, wherein the stent comprises an antibacterial coating.

19. The bioresorbable tubular organ stent assembly of claim 18, wherein the antibacterial coating comprises one or more of cellulose, silver, or a resorbable polymer coating infused with an antibiotic compound.

20. The bioresorbable tubular organ stent assembly of claim 1, wherein the stent comprises a porous structure comprising interconnected pores.

21. The bioresorbable tubular organ stent assembly of claim 1, wherein the stent comprises one or more of magnesium (Mg), zinc (Zn), iron (Fe), tungsten (W), molybdenum (Mo), polylactic acid (PLA), polyglycolide (PGA), poly(lactic-co-glycolic acid) (PLGA), polyethylene glycol (PEG), polycaprolactone (PCL), poly(glycerol sebacate) (PGS), alginate (all forms), cellulose (all forms), chitosan (all forms), hyaluronic acid (human and animal derived), or collagen (human and animal derived types 1, 2, 3, 4, and 5.

22. A method of connecting sections of a tubular organ, the method comprising:
inserting a first end portion of a bioresorbable stent into an open end of a first section of the tubular organ;
inserting a second end portion of the bioresorbable stent into an open end of a second section of the tubular organ; and
securing the first section and the second section to the bioresorbable stent.

23. An intestinal stent assembly comprising:
an intestinal stent configured for retaining a first section of an intestine in contact with a second section of the intestine to accommodate joining of the first section and the second section via healing, wherein a first end portion of the intestinal stent is configured to accommodate insertion into the first section of the intestine and inhibit removal from the first section of the intestine, wherein a second end portion of the intestinal stent is configured to accommodate insertion into the second section of the intestine and inhibit removal from the second section of the intestine, and wherein the
intestinal stent is made from one or more bioresorbable materials; and
one or more retention devices configurable to secure the first section of the intestine and the second section of the intestine to the intestinal stent, wherein each of the one or more retention devices is made from one or more bioresorbable materials.
